(19) United States
(12) Patent Application Publication
(10) Pub. No.: US 2010/0222809 A1
(43) Pub. Date: Sep. 2, 2010

(54) Portable Hands-Free Device to Locate and Release Active Trigger Points That Develop in Muscles

(76) Inventor: Michele Jayne Grabill, Black Mountain, NC (US)

Correspondence Address: DAVID M. CARTER;CARTER, SCHNEDLER & WARNOCK, P.A., P.O. BOX 2985, ASHEVILLE, NC, 28802, US

(21) Appl. No.: 12/711,677

(22) Filed: Feb. 24, 2010

Related U.S. Application Data
(60) Provisional application No. 61/156,017, filed on Feb. 27, 2009.

(51) Int. Cl. A61B 17/00

(52) U.S. Cl. 606/204

(57) ABSTRACT

There is hereby provided a trigger point therapy device including a height adjustable bracket having a plurality of vertical holes, and an extension bar, which is received in one of the vertical holes.

### RELATIONSHIP TO PRIOR APPLICATION

This is a U.S. non-provisional application relating to and claiming the benefit of U.S. Provisional Patent Application Ser. No. 61/156,017, filed Feb. 27, 2009.

### BACKGROUND

This invention relates to devices which release trigger points that develop in muscles. In particular, it relates to devices which enable the self-treatment of such trigger points.

Active trigger points often develop in muscles for various reasons including over use. These trigger points promote tightness and pain. Most often, trigger points develop in a person's back muscles. Treatment to alleviate the pain of trigger points is often done by massage therapists and physical therapists who apply pressure to the trigger points. There are home remedies which include the positioning of a ball, such as a tennis ball, between a patient's back and a wall with the patient moving the ball with his or her back so as to apply pressure to the trigger points.

Patent Publication No. US 2008/0139981 shows a trigger point therapy device consisting of a mounting bracket which is affixed to a wall by the use of screws which permanently attach the mounting bracket to vertical studs which are associated with the wall. The bracket includes a vertical rod having a plurality of holes therein. The holes receive a probe which can be moved to various heights, to the level of the user's trigger points.

### SUMMARY OF THE INVENTION

In one embodiment of the present invention there is provided a trigger point therapy device including an adjustable bracket adapted to be removably mounted to a portion of a door frame. The bracket includes a clamp for securing the bracket to the door frame. The bracket further includes a plurality of holes. The holes are substantially horizontal with each hole being located at different heights when the bracket is mounted to the door frame. An extension bar is provided. A first portion of the extension bar is selectively and removably received in one of the holes. A second portion of the extension bar is adapted to make therapeutic contact with a user's trigger point. The height of the extension bar is readily adjusted by moving the bracket and by selectively placing the extension bar in a particular hole.

In accordance with another form of this invention there is provided a trigger point therapy device including an adjustable bracket adapted to be removably mounted to a portion of a door frame. The bracket includes a clamp for securing the bracket to the door frame. The bracket further includes at lease one hole. The hole is substantially horizontal when the bracket is mounted to the door frame. An extension bar is provided. A first portion of the extension bar is selectively and removably received in the hole. A second portion of the extension bar is adapted to make therapeutic contact with a user's trigger point. The height of the extension bar is readily adjusted by moving the bracket.

### BRIEF DESCRIPTION OF THE DRAWINGS

FIG. 1 is a rear perspective view showing the adjustable bracket of the subject invention.

FIG. 2 is a front perspective view of the adjustable bracket of FIG. 1.

FIG. 3 is a side perspective view of the adjustable bracket of FIG. 1.

FIG. 4 is a front elevational view of the adjustable bracket of FIG. 1 shown mounted to a door frame.

FIG. 5 is rear elevational view of the adjustable bracket of FIG. 1 shown mounted to a door frame.

FIG. 6 is a side elevational view of an extension bar having a single prong which may be used with the bracket of FIG. 1.

FIG. 7 is a side elevational view showing an alternative embodiment of the extension bar of FIG. 6.

FIG. 8 is a side elevational view of an extension bar having two prongs.

### DESCRIPTION OF THE PREFERRED EMBODIMENTS

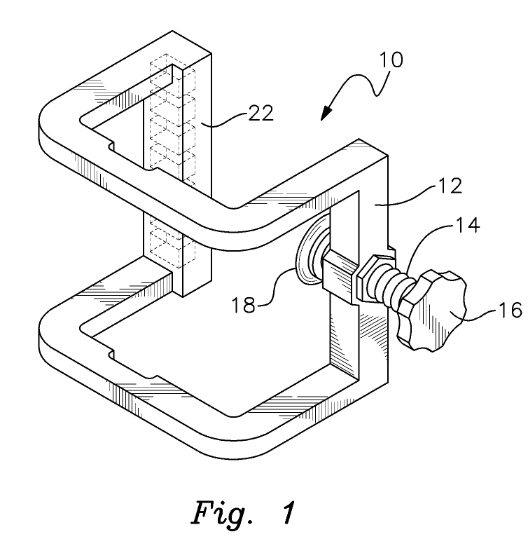

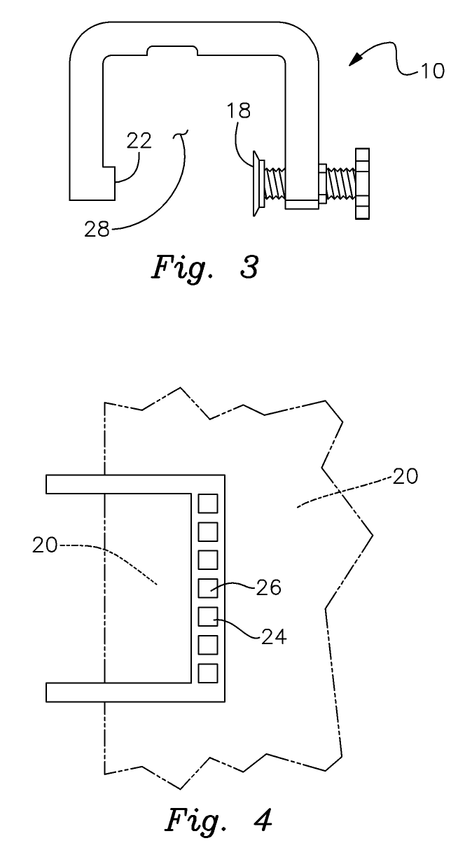

Turning now more particularly to FIGS. 1-8, there is provided adjustable bracket 10, including “C” shaped frame 12. A threaded hole is provided in “C” shaped frame 12 for receiving worm screw 14. Worm screw 14 includes handle 16. Plate 18 is attached to the end of worm screw 14. The worm screw enables bracket 10 to be clamped to a door frame and to permit the bracket 10 to be moved upwardly and downwardly, depending on the height of the user as well as the location of the trigger point, which is normally on the user's back.

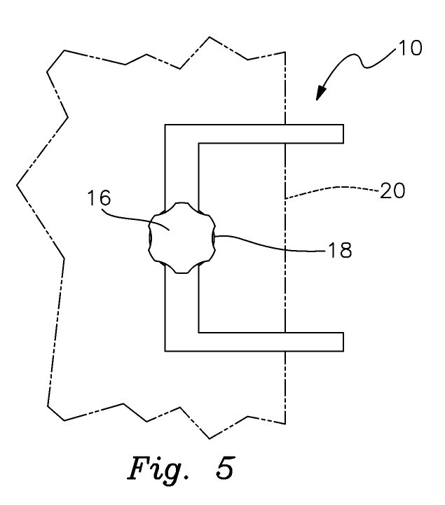

As can be seen from FIG. 5, plate 18 makes contact with door frame 20 to secure adjustable bracket 10 in place. Stop 22 makes contact with the other side of door frame 20 to complete the clamping.

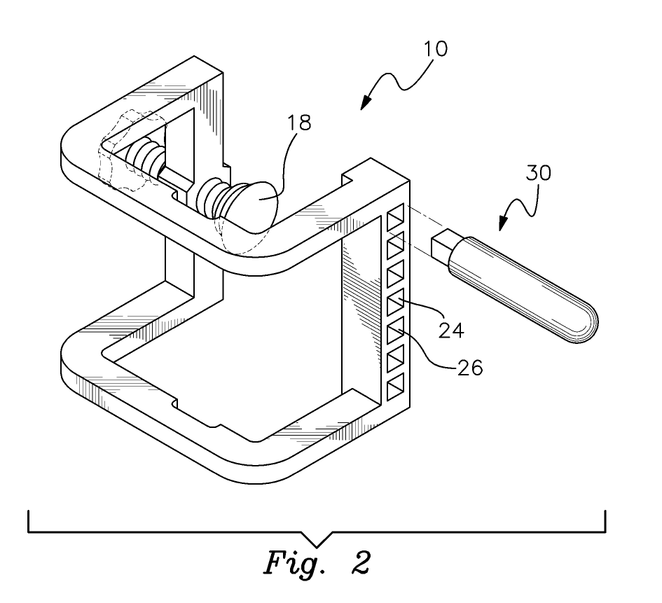

As can be seen from FIG. 2, a plurality of holes 24 and 26 are provided in bracket 10. The holes are substantially horizontal when the bracket is mounted to the door frame. Also, when the bracket is mounted to the door frame, each hole is located at a different height. The adjustable bracket is somewhat “C” shaped which provides open space 28 so as to receive door frame 20.

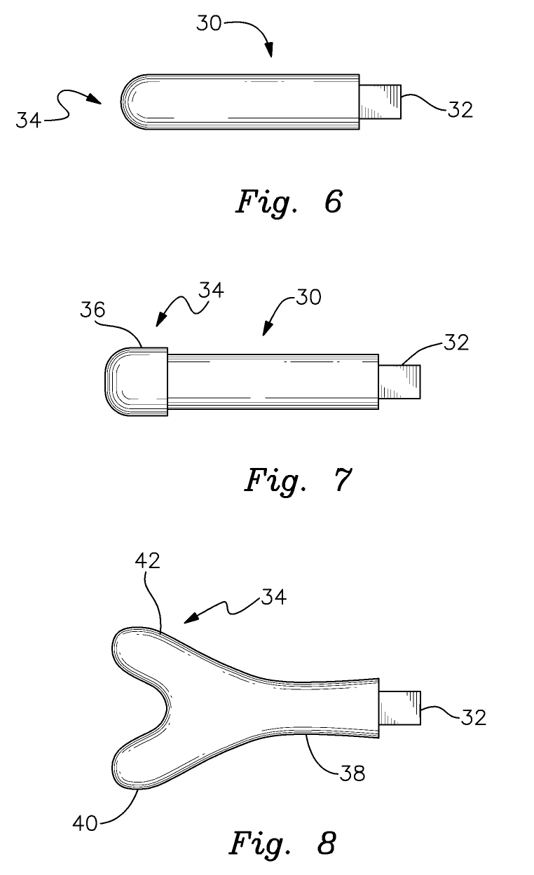

A complete trigger point therapy device includes bracket 10 and an extension bar. Referring now more particularly to FIGS. 6 and 7, extension bar 30 includes first portion 32 and second portion 34. First portion 32 is adapted to be received in one of the holes in the adjustable bracket 10, such as for example, holes 24 or 26. Thus, once the first portion of the extension bar has been placed in one of the holes in the adjustable bracket 10, the extension bar 30 projects away from the adjustable bracket and thus the door frame so that the second portion 34 of the extension bar 30 may make therapeutic contact with the user's trigger point. In the embodiment of FIG. 7, rubber tip 36 is placed over the second portion 34 of extension bar 30 to provide comfort for the user.

FIG. 8 shows extension bar 38, which is similar to extension bar 30 shown in FIG. 6, except that the second portion of extension bar 38 includes a pair of prongs, namely prongs 40 and 42.

The trigger point therapy device of the subject invention is both portable and highly adjustable. The height of the extension bar is readily adjusted by moving the bracket up or down the door frame and by selectively placing the extension bar in a particular hole. Preferably, holes 24 and 26, as well as the first portions of the extension bars are square.

When the trigger point therapy device is not in use, the extension bars may be stored in the “C” shaped opening 28 of the adjustable bracket 10 using metal magnetic storage buttons.

The adjustable bracket 10 and the extension bars 30 and 38 are designed to locate and provide pressure to any trigger point or area of soft tissue tension. The trigger point therapy device is hands free and effectively de-sensitizes trigger points that often exist in soft tissue resulting in tightness and pain by bombarding the trigger point with a steady stream of stimuli until the trigger point relaxes. The trigger point therapy device may be easily located over any area of the body by leaning against the extension bar and applying pressure. The device may also be used in a standing or sitting position and is effective from neck to ankle by simply moving it upwardly or downwardly on the door frame and by making fine tune adjustments by moving the extension bar into the appropriate hole in the adjustable bracket. The device is light weight and easily portable and may be used at any location that has a door frame, including home, office, training room or hotel room. It may be used by anyone, such as athletes, secretaries, hair stylists, home makers, or surgeons. It is an excellent method of relieving tension brought about by a long day of driving or sight-seeing while traveling.

In one embodiment, the adjustable bracket and the extension bars are made of wood, the magnetic storage buttons are made of metal and the extension bar tips are made of rubber. In order to reduce costs, it is preferred that the extension bars and adjustable bracket be made of molded plastic.

The adjustable bracket 10 is attached to door frame 20 by simply turning arm screw handle 16 in a clockwise direction until screw plate 18 and stop 22 are tightly seated against door frame 20. The interchangeable extension bars 30 and 38 are then removed from storage and inserted into the appropriate hole in the bracket 10 such as 24 or 26. The adjustable bracket may then be moved upward or downward to the desired height on door frame 20 to meet the trigger point. The user, standing or sitting, applies pressure on the trigger point by leaning gently against the second portion 34 of extension bar 30 or 38. This position is maintained until the involved soft tissue relaxes. If the height of the extension bar is not appropriate for a particular trigger point, the user simply moves the extension bar upwardly or downwardly on the adjustable bracket by using the appropriate hole such as hole 24 or 26. The adjustable bracket 10 may be removed from door frame 20 by simply turning screw handle 16 in the counter-clockwise direction to loosen screw plate 18 and stop 22 from door frame 20.

While the invention has been described in terms of the above embodiments, those skilled in the art will recognize that the invention can be practiced with modification within the spirit and scope of the appended claims.

1. A trigger point therapy device comprising:
an adjustable bracket adapted to be removably mounted to a portion of a door frame; the bracket including a clamp for securing the bracket to the door frame; the bracket further including a plurality of holes; the holes being substantially horizontal with each hole being located at a different height when the bracket is mounted to the door frame;
an extension bar; a first portion of the extension bar selectively and removably received in one of the holes; a second portion of the extension bar adapted to make therapeutic contact with a user's trigger point; whereby the height of the extension bar is readily adjusted by moving the bracket and by selectively placing the extension bar in a particular hole.

2. A trigger point therapy device as set forth in claim 1 wherein the clamp includes a worm screw.

3. A trigger point therapy device as set forth in claim 1 wherein the second portion of the extension bar has one prong.

4. A trigger point therapy device as set forth in claim 1 wherein the second portion of the extension bar includes at least two prongs.

5. A trigger point therapy device as set forth in claim 1 wherein the adjustable bracket and the extension bar are portable.

6. A trigger point therapy device comprising:
an adjustable bracket adapted to be removably mounted to a portion of a door frame; the bracket including a clamp for securing the bracket to the door frame; the bracket further including at least one hole; the hole being substantially horizontal when the bracket is mounted to the door frame;
an extension bar; a first portion of the extension bar being selectively and removably received in the hole; a second portion of the extension bar adapted to make therapeutic contact with a user's trigger point; whereby the height of the extension bar is readily adjusted by moving the bracket.

7. A trigger point therapy device as set forth in claim 6 wherein the clamp includes a worm screw.

8. A trigger point therapy device as set forth in claim 6 wherein the second portion of the extension bar has one prong.

9. A trigger point therapy device as set forth in claim 6 wherein the second portion of the extension bar includes at least two prongs.

10. A trigger point therapy device as set forth in claim 6 wherein the adjustable bracket and the extension bar are portable.
